(19) United States
(12) Patent Application Publication
(10) Pub. No.: US 2026/0226427 A1
(43) Pub. Date: Aug. 6, 2026

(54) TEST SYSTEM COMPRISING STABLY CYP-TRANSFECTED OR TRANSDUCED MAMMALIAN CELLS

(71) Applicants: BRANDENBURGISCHE TECHNISCHE UNIVERSITÄT COTTBUS-SENFTENBERG, Cottbus (DE); FRAUNHOFER-GESELLSCHAFT ZUR FÖRDERUNG DER ANGEWANDTEN FORSCHUNG E.V., München (DE)

(72) Inventors: Jan-Heiner KÜPPER, Kleinkoschen (DE); Christian SCHULZ, Kleinkoschen (DE)

(21) Appl. No.: 19/149,449

(22) PCT Filed: Jan. 31, 2024
(86) PCT No.: PCT/EP2024/052305

§ 371 Date: Jul. 18, 2025

(30) Foreign Application Priority Data
Jan. 31, 2023 (DE) 10 2023 102 366.7

(51) Int. Cl. C12N 9/02; C12Q 1/26

(52) U.S. Cl. CPC C12N 9/0042; C12N 9/0071; C12Q 1/26; C12Y 106/02004

(57) ABSTRACT

The present invention relates to a mammalian cell or cell line, preferably a CHO cell, containing polynucleotides stably integrated into the genome after transformation, encoding at least one P450 cytochrome (CYP) and NADPH-P450 cytochrome (CPR, POR), their use, and a test system obtainable therefrom, preferably by providing microsomes (fractions) or suspension cells.

The invention relates to a mammalian cell or cell line, preferably a CHO cell, containing polynucleotides stably integrated into the genome after transfection or transduction, encoding at least one cytochrome P450 (CYP) and NADPH cytochrome P450 oxidoreductase (CPR, POR), their use and a test system obtainable therefrom, preferably by providing microsomes (fractions) or suspension cells.

The liver is a central detoxification organ of the metabolism. Liver cells (hepatocytes) represent 70-80% of all cells in the liver and perform important physiological liver functions (Elaut et al. (2006)).

The liver uses biotransformation or metabolism to excrete or detoxify ingested substances (e.g., drugs, toxins, natural substances). The phase I enzymes of the cytochrome P450 (CYP450) system are particularly important for biotransformation. CYP450 enzymes are oxidoreductases that cause oxidative degradation or metabolism of numerous substances, including drugs. Of the numerous CYP450 isoenzymes with different substrate specificities that exist in humans, the isoenzymes CYP1A2, -2C9, -2C19, -2D6, -2E1, and -3A4 alone are responsible for approximately 90% of all oxidative metabolism of drugs. In many cases, it is only through these biochemical changes that a large number of drugs obtain their curative efficacy or even cause toxic metabolites with undesirable drug effects. For the most part, the metabolism of foreign substances takes place via biotransformation mechanisms involving corresponding enzyme and transport systems in the liver. Metabolization reactions of active substances/foreign substances in phase 1 biotransformation, which are the subject of the invention described, are predominantly carried out by enzymes of the cytochrome P450 family (CYPs), which are located in the endoplasmic reticulum (ER). All metabolization reactions catalyzed by CYPs are dependent on NADPH cytochrome P450 oxidoreductase (CPR, POR), which is also located in the ER and, as an essential cofactor, sequentially transfers electrons from NADPH to CYPs.

Determining which CYPs are involved in the metabolism of an active substance, which metabolites are formed by the respective CYP, and how these are further metabolized by other CYP-dependent reactions is always one of the initial steps in the development of new drugs. For the precise elucidation of metabolites and their provision in sufficient quantities for further analysis, ADME test systems (absorption, distribution, metabolism, excretion) are particularly necessary, as they provide reliable information on CYP-specific metabolic reactions. Due to the complexity of these processes, it is sometimes difficult to identify the CYPs involved and thus predict metabolic processes and interactions in the human liver.

In toxicological in vitro drug studies, human test systems such as cell cultures from primary cells and cancer cell lines with various modifications are primarily used in order to replicate processes in the human liver as closely as possible. Subcellular isolates of the ER, known as microsomes, are also important for phase I studies, as CYPs (including the cofactor CPR, POR) are mostly located in this cell compartment. A major disadvantage of these systems with regard to the identification and characterization of specific CYP metabolisms is their lack of specificity, since the physiological CYP population expressed and active in liver cells consists of several different CYPs. Furthermore, the activity of some relevant CYPs may be too low for potential drug candidates, meaning that the metabolites formed cannot be detected, let alone produced in sufficient quantities for further studies.

In ADME studies to elucidate CYPs involved in drug metabolism, microsomes from recombinant expression systems are currently the most commonly used. Established models include recombinant microsomes with human CYPs (including cofactors) from baculovirus-infected insect cells (known as “supersomes” or “baculosomes”-BD Biosciences, Corning, ThermoFisher, Merck), from E. coli-based expression systems (so-called Cypex Bactosomes as “Classic Bactosomes” and “EasyCYPs”-Sekisui XenoTech) and dried preparations of the yeast Pichia pastoris (known as “CypExpress” from Oxford Biomedical Research or “EZCyp” from BioVision). In addition, preparations of recombinant expression of microbial CYPs from actinomycetes in E. coli are also commercially available (known as PolyCYPs—Hypha Discovery). The insect model based on baculovirus infection is particularly characterized by high enzyme activity that is dependent on the respective CYP expressed and can be considered the current gold standard. All of the expression systems described have in common that the cell models used for the expression of human enzymes are not mammalian-based, and therefore relevant differences between the recombinantly expressed CYPs and human enzymes and their function cannot be ruled out. Furthermore, the relevant literature does not conclusively clarify whether the cell models used for recombinant CYP expression exhibit non-specific (CYP) background activities relevant to the metabolism of an active substance at the cellular and microsomal level. The insect cell model, on which scientific opinion is divided, can be cited as an example here. Brandon et al. point out in a review published in 2003 that insect cells do not exhibit endogenous CYP activity. In contrast, Nauen et al. describe existing endogenous CYP background activity in the baculovirus insect cell expression system in a review article published in 2021. Sf9 cells in particular exhibit basal detoxification activity for some substance classes. This is said to be significantly lower in the High Five insect cell line (cabbage moth ovarian cells; cell line: BTI-TN-5B1-4). Either non-specific CYP background activity of the control supersomes (FDA study NL0060848 from 2005 on the metabolism of asenapine) or significant differences in the conversion kinetics of active substances are observed when comparing insect cell-based CYP3A4 microsomes (CYP3A4 supersomes) with microsomes produced from human liver epithelial cells (with recombinant CYP3A4 expression) (Christensen et al. 2009). Furthermore, due to the lytic culture system after Baculovirus infection according to, the established insect cell models at the cellular level offer only limited possibilities for long-term cultures.

Recombinant expression systems of human CYPs with mammalian cell models have been described, including studies with heterologous expression of human CYPs (including cofactors) in human HEK293FT kidney epithelial cells (Kumondai et al. 2020) and in lung (V79 cells: Jacob et al. 1996; Krebsfänger et al. 2003; Scheuenpflug et al. 2005) and ovarian cells of Chinese hamsters (CHO-DUKX-B11 cell line: Ding et al. 1997 &amp; 2001, Deeni et al. 2013 and a trade fair poster CXR Biosciences, Deeni 2009). In none of the mammalian models described were the human CYP genes and cofactors specifically integrated into the genome in a stable manner so that they are present in the cell nucleus as an episomal recombinant viral genome after transfection or infection with modified adenoviruses, for example. Furthermore, the mammalian cell lines modified with human CYPs are adherent and serum-dependent cell culture models. Both factors limit the possibilities for upscaling and pharmaceutical/therapeutic use of metabolites.

There is therefore a great need for a test system with no or very low endogenous CYP background for testing substances, in particular xenobiotics, drug candidates, and drugs as test substances, especially the interaction of a test substance with at least one CYP.

The invention therefore has the following objectives, in particular the provision of a suitable test system which a.) Cytochrome P450 (CYP) and NADPH-Cytochrome P450 oxidoreductase (CPR, POR) in sufficient quantity and quality in overexpression, and b.) has as little as possible to no endogenous CYP background activity of an expression system.

The tasks are essentially solved by at least one patent claim.

Therefore, the invention relates to a mammalian cell or a cell line obtainable therefrom containing, after transfection or transduction, polynucleotides stably integrated into the genome encoding at least one cytochrome P450 and NADPH-cytochrome P450 oxidoreductase, wherein the polynucleotides are preferably introduced into the mammalian cell with at least one expression vector.

In a preferred embodiment of the invention, the test system comprises mammalian cell lines with at least one cytochrome P450 and NADPH cytochrome P450 oxidoreductase, which are provided as a suspension culture in high cell density of on average 5×10⁶ cells/mL with >80% vitality. The methods for measuring cell numbers and vitality are known to those skilled in the art. From the mammalian cells according to the invention, microsomal fractions are obtained by methods known to those skilled in the art, which, based on 1×10⁷ starting cells, advantageously have a protein yield of at least 100 μg (protein quantity or enzyme quantity) and more, in particular at least 200 μg (protein quantity or enzyme quantity) or at least 300 μg (protein quantity or enzyme quantity). Due to the high protein yield, a very well measurable conversion of CYP enzyme activity or the metabolites formed is possible. As an example, a conversion with the substance phenacetin in of a mammalian cell with CYP1A2 and CPR shows a phenacetin metabolite production of 17 μg after 2 hours and 101 μg after 48 hours.

The sensitivity limit of the test system according to the invention for CYP-specific enzyme activity in conversion with microsomes from mammalian cells according to the invention with at least one cytochrome P450 and NADPH cytochrome P450-oxidoreductase is a maximum of 1-10 μg of microsomal protein in a 50 μL reaction and 10 min reaction time with a P450 Glo assay. In this assay known to those skilled in the art (e.g., P450 Glo Assays from Promega), a substrate specific for the respective CYP is provided and the CYP enzyme activity can be detected by means of a luminescence reaction. The detection of a metabolite formed via CYP (e.g., metabolite of a drug) can be carried out, for example, using the HPLC analysis method known to those skilled in the art. The sensitivity limit for the formation of such a metabolite in the test system according to the invention is preferably 30-300 μg of total microsomal protein in a 200 μL test reaction and 30 min reaction time.

It is still preferred that the mammalian cell is not a liver cell or non-hepatic cell and is selected from the group HEK293 cells, CHO cells, Hela cells, BHK cells, L929 cells or respective cell lines, as well as mammalian cell lines from the ZKBS list that are not derived from hepatocytes (cell line list of the Central Commission for Biological Safety: https://zag.bvl.bund.de/zelllinien/index.jsf;jsessionid=af9u6CdFQWhIMTatrjeI47SX_vzdBRAe8bzcchSe.subs208?dswid=1187&amp;dsrid=42).

According to the invention, CHO (Chinese hamster ovary) cells and cell lines obtained therefrom are particularly preferred, since they advantageously have no or low endogenous CYP background activity and, consequently, the at least one cytochrome P450 (CYP) introduced by stable transfection or transduction is present without any relevant background activity of endogenous CYPs. The enzyme activity is advantageously enhanced by the further introduced CPR.

Low or no background activity of endogenous CYPs is present if, using the HPLC method for detecting metabolites formed, this is below the detection limit and/or a maximum of 1-5%, preferably 1% of the specific activity in pmol/mg/min for at least one introduced cytochrome P450 and NADPH cytochrome P450 oxidoreductase.

The background activity of endogenous CYPs in the parental mammalian cells can be determined prior to the stable introduction of the CYP/CPR complex into the mammalian cell according to the invention. The parental mammalian cells can serve as a negative control.

Furthermore, it is preferred that mammalian cells or the corresponding cell line are obtained as suspension cells or as microsomes.

The term microsome also includes microsome fractions and can be obtained by processing the mammalian cells or the corresponding cell line provided. Microsome and microsome fraction are therefore to be read as synonyms.

Within the scope of this invention, the polynucleotides for cytochro e and NADPH-cytochro e oxidoreductase can be obtained from the usual databases. For example, gene sequences can be obtained from databases of the National Center for Biotechnology (NCBI) and the National Library of Medicine (NLM). Human cytochrome P450 and NADPH cytochrome P450 oxidoreductase polynucleotide sequences are particularly preferred. The databases mentioned contain, for example, cDNA sequences for CPR (NM 000941.2), CYP1A2 (BC067428.1), CYP2B6 (BC067430.1) or CYP3A4 (NM 017460.6), but also for other CYPs relevant for biotransformation, which can be introduced into an expression vector.

TABLE 1
mRNA IDs and primer
sequences of human target genes

| Gene | | | |
|---|---|---|---|
| Target | | Primer sequences | |
| genes | mRNA ID | (5′→3′) | |
| CPR | NM_000941.2 | fw | AAGGCGGTGCCC |
| | | | ACATCTAC |
| | | rev | TAGCGGCCCTTG |
| | | | GTCATCAG |
| CYP1A2 | BC067428.1 | fw | CTGGAGACCTTC |
| | | | CGACACTC |
| | | rev | AGGGCTTGTTAA |
| | | | TGGCAGTG |
| CYP2B6 | BC067430.1 | fw | CTCTCCATGACC |
| | | | CACACTAC |
| | | rev | TGTTGGGGGTAT |
| | | | TTTGCCCA |
| CYP3A4 | NM_017460.6 | fw | GTGGGGCTTTTA |
| | | | TGATGGTCA |
| | | rev | GCCTCAGATTTC |
| | | | TCACCAACACA |

Within the scope of this invention, cytochrome P450 or CYPs may be used which are expressed in primary human liver cells and other cells that are relevant for the biotransformation of drugs and xenobiotics or will become relevant in the future, and are selected from the group CYP1A1, CYP1A2, CYP1B1, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3A4, CYP3A5, CYP3A7, CYP3A43, CYP4A11, CYP4A22, CYP4B1, CYP4F2, CYP4F3, CYP4F8, CYP4F11, CYP4F12, CYP4F22, CYP4V2, CYP4X1, CYP4Z2, CYP5A1, CYP7A1, CYP7B1, CYP8A1, CYP8B1, CYP11A1, CYP11B1, CYP11B2, CYP17A1, CYP19A1, CYP20A1, CYP21A1, CYP21A2, CYP24A1, CYP26A1, CYP26B1, CYP26C1, CYP27A1, CYP27B1, CYP27C1, CYP39A1, CYP46A1, CYP51A1.

The aforementioned mammalian cells, cell lines, suspension cells or microsomes obtained according to the invention can be mixed with at least one test substance, and the metabolites can be identified and characterized.

The test system therefore serves to detect metabolites, in particular due to the specific CYP test substance interaction, whereby an enzymatic conversion of the test substance takes place with one or more CYP450 enzymes that have been stably introduced via transfection or transduction, and specific metabolites can be obtained. The use of a test system with microsomes is particularly preferred.

A further advantage of the test system according to the invention is that, due to low or completely absent background enzyme activity of endogenous CYP450 enzymes, the test system is suitable for identifying or characterizing the responsibility or function of the CYP450 enzyme(s) that have been stably introduced by transfection or transduction for the metabolic conversion of chemical substances, xenobiotics, or drugs.

The invention therefore relates to a test system for detecting an interaction of at least one test substance with at least one CYP450 enzyme.

The invention therefore relates to a test system for detecting the biotransformation of at least one test substance by at least one CYP450 enzyme responsible for the biotransformation, wherein an enzymatic conversion of at least one test substance with one or more CYP450 enzymes takes place.

Another advantage of the test system according to the invention is that the inhibition or activation of the CYP450 enzyme or enzymes that have been stably introduced by transfection or transduction can also be investigated by at least one test substance without a background of endogenous CYP450 enzymes. This makes it possible to advantageously investigate interactions between drugs with regard to metabolic conversion by a specific CYP450 enzyme and activation or inhibition of another CYP450 enzyme.

The test system is particularly suitable for performing ADME tests (supra) in vitro. ADME describes the processes that the body performs on the active ingredients contained in a drug after administration, particularly with regard to toxicology.

Therefore, the invention also relates to a method for carrying out the aforementioned test systems according to the invention or the use of such a test system according to the invention for carrying out a method, in particular wherein an enzymatic conversion of at least one test substance with one or more CYP450 enzymes, which have been stably introduced by transfection or transduction, takes place.

Therefore, the invention also relates to a screening method for detecting a test substance, wherein an enzymatic conversion of at least one test substance with one or more CYP450 enzymes, which have been stably introduced by transfection or transduction, takes place.

The test system according to the invention can be used particularly advantageously for long-term studies, since it is possible to simulate the human liver almost completely with regard to biotransformation. Based on the identified metabolites, side effects of a drug can be determined, for example, by producing the metabolite formed in a suitable quantity and testing it toxicologically on target cells or organs.

The test substance contains at least one chemical substance, a mixture of substances, in particular at least one drug or active ingredient or a xenobiotic. The chemical substances are preferably organic molecules which, in addition to carbon (C) and hydrogen (H), may contain heteroatoms such as oxygen (O), nitrogen (N), sulfur(S), or phosphorus (P). The chemical substances may have linear and/or ring-shaped carbon chains including heteroatoms. Organic molecules with less than 1,500 g/mol, in particular less than 750 g/mol, less than 500 g/mol, less than 250 g/mol, are preferred. Furthermore, it is preferred that at least one chemical substance contains at least one chiral carbon atom.

The substances obtained from the test substances or the metabolites obtained in the presence of one or more enzymes can be sufficiently analyzed, for example, by means of combined analytical methods such as GC/LC-MS, IR, NMR, and in particular can be routinely subjected to structural elucidation.

An “enzyme” within the meaning of this invention is a protein, namely at least one selected cytochrome P450 (CYP) or a complex of cytochrome P450 (CYP) and NADPH cytochrome P450 oxidoreductase (CPR, POR), which can catalyze one or more biochemical reactions. An enzyme according to the invention is capable of obtaining a first (enzyme) product or products, namely metabolites, from a test substance (substrate). In particular, a metabolite profile can be created.

The “specific activity” of an enzyme within the meaning of this invention is the amount of a substrate metabolite (in pmol) formed by an enzyme per protein (mg) used in a given time (minute). In the case of cells or microsomes, if no specific purified protein is available, the total protein is used as the reference value.

The invention also encompasses transgenic mammalian cells produced by means of viral vectors (e.g., lentiviruses, e.g., DE 69830663 T2). The production of such mammalian cells is known to those skilled in the art.

Preferred suitable promoters for expression vectors are, but are not limited to, EF-1, SV-40, CMV, RSV, CAG, and many others, including conditionable, i.e., inducible promoters such as TET on, -off, Cumat-inducible promoter, dexamethasone-inducible promoter, ecdysone-inducible promoter, T7 RNA polymerase-inducible promoter, light-dependent promoters or stress-inducible promoters (e.g. temperature) as well as with the help of auxiliary sequences such as IRES (internal ribosome entry site), sequences for antibody detection and protein purification such as V5 tag, Flag tag, c-myc tag, His tag, GST tag, Strep tag, GFP, LacZ, luciferase, etc., sequences for protein cleavage of such tags such as picornavirus 2A protease, sequences for thrombin-mediated cleavage, etc. Such promoters and auxiliary sequences are well known to those skilled in the art.

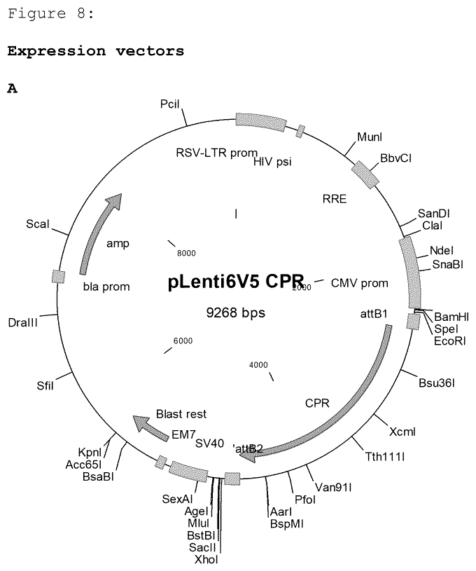

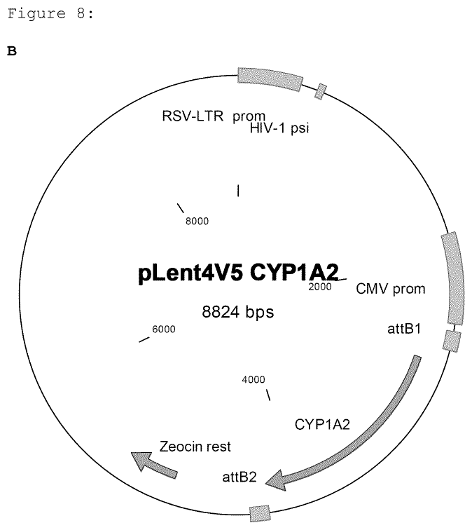

Examples of expression vectors are shown in FIG. 8.

The test system may comprise a conventional medium, culture medium, culture fluid, and in particular may contain further cofactors. Suitable cell cultures and culture media are known to those skilled in the art and are commercially available. Preferably, a cell suspension can be provided according to the invention.

The test system can be fixed in vitro on a solid carrier. Synonymously, one can speak of an assay.

The following examples and figures serve to explain the invention in more detail, without however limiting the invention to these examples and figures.

### EXAMPLES AND FIGURES

### Example 1

### Generation of Mono-CYP CHO Cell Lines

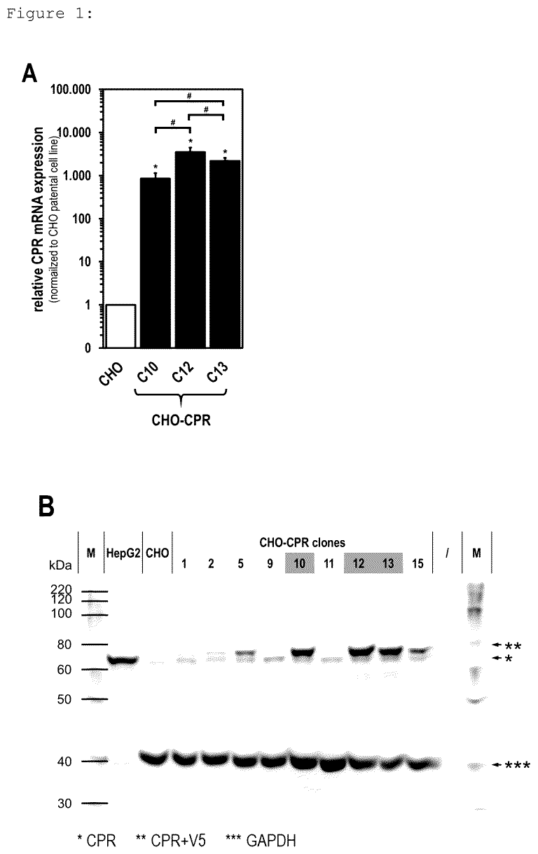

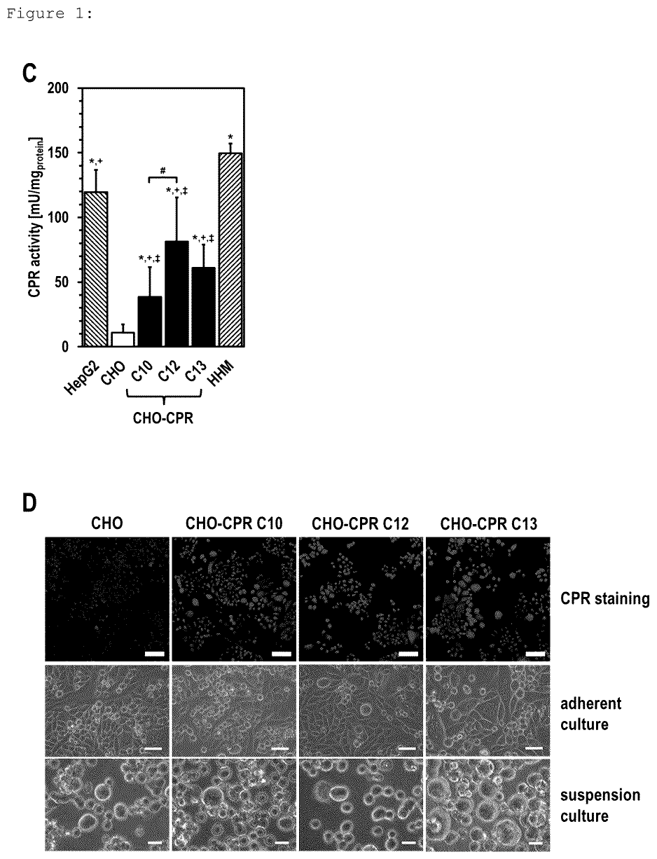

The generation of mono-CYP CHO cell lines was carried out in a two-step modification process. Initially, the CHO-K1 parental cell line (serum-free suspension culture) was stably genetically modified by lentiviral gene transfer with the human cDNA for NADPH cytochrome P450 oxidoreductase (CPR, POR) under the control of a CMV promoter. CPR is an essential cofactor for CYP enzyme activity, as it exclusively enables the sequential electron transfer of NADPH to CYP enzymes. The increased expression and activity of human CPR in selected CHO-CPR clones was characterized, and the most suitable clone 12 was selected for further genetic modification with human CYP enzymes (FIG. 1).

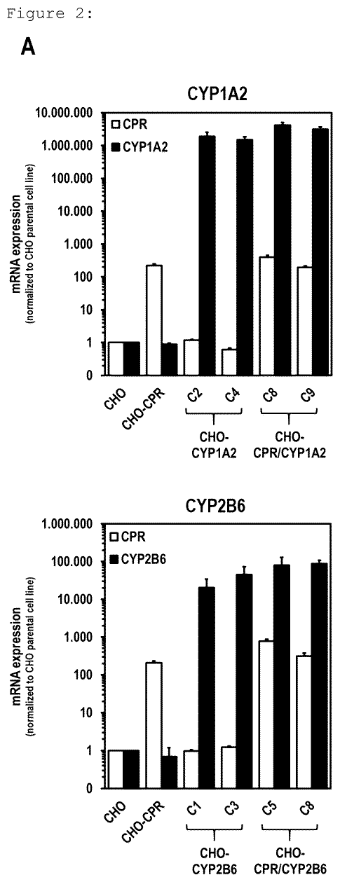

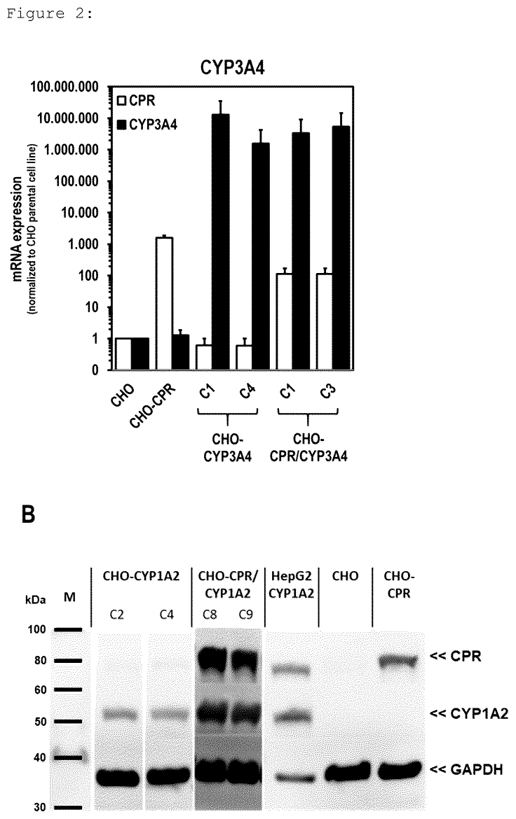

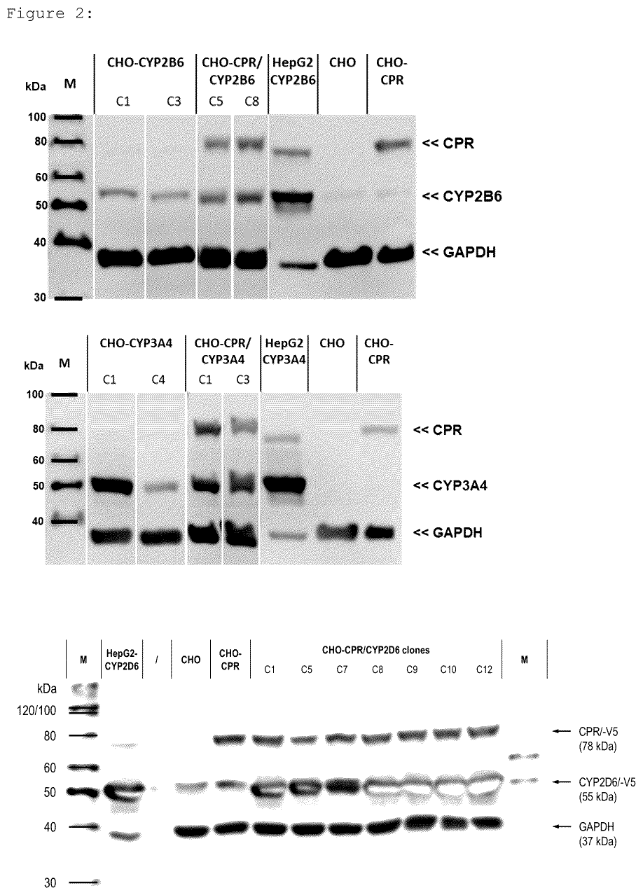

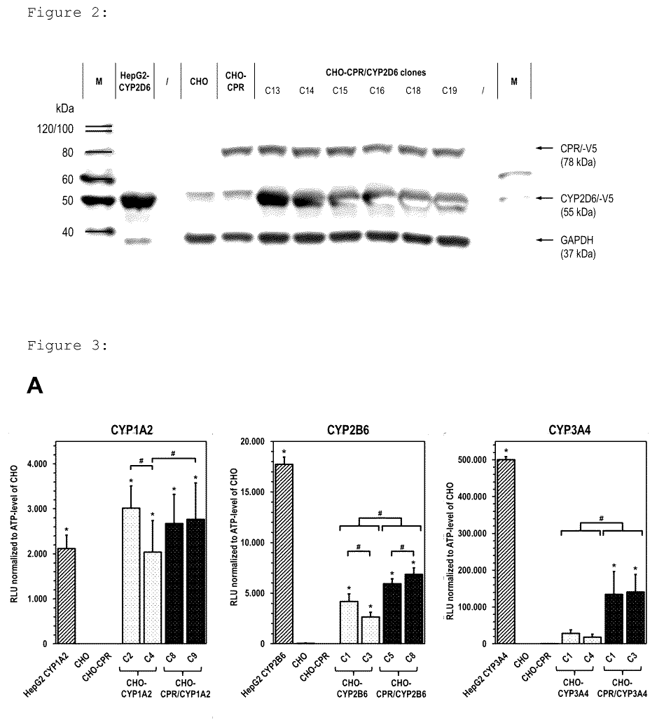

In the second step of the modification process, the most suitable CHO-CPR clone 12 was again equipped with the human CYP cDNA of CYP1A2, CYP2B6, CYP3A4, CYP2D6, or CYP2C19 using lentiviral gene transfer. These cDNAs are regulated by a CMV promoter in the same way as CPR. To enable comparative experiments on the CPR dependence of different CYPs, the CHO-K1 parental cell line without increased CPR activity was also equipped with the respective CYP CDNAs. Subsequent characterization and selection of the best-performing cell clones showed the expected specific CYP expression compared to the parental cell lines CHO-K1 and CHO-CPR (FIG. 2). Only for CYP2B6 and CYP2D6 was a weak protein band also detected in the parental cell lines.

### Example 2

### Specific CYP Activity of Mono-CYP CHO Cell Clones in a Cellular Suspension Culture Model

### Commercial CYP Activity Assays (Glo Assays-Promega)

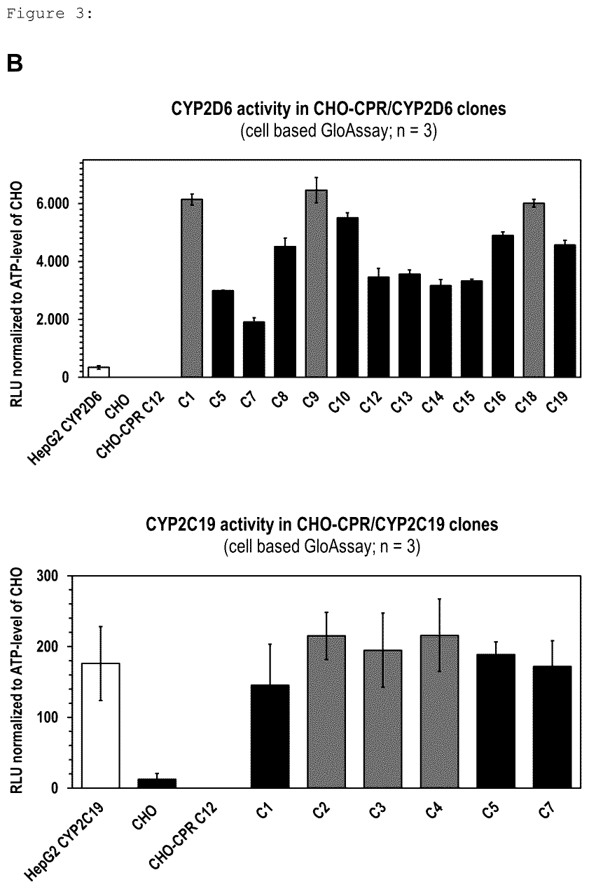

Initial enzyme activity studies with the selected mono-CYP CHO cell lines (with and without artificial human CPR) using commercial kits indicated specific mono-CYP activity in all CHO-CYP clones. No background activity was detected in the parental cell lines CHO-K1 and CHO-CPR clone 12 for any of the CYPs investigated (FIG. 3).

### Example 3

Conversion Studies with Prototype Substances

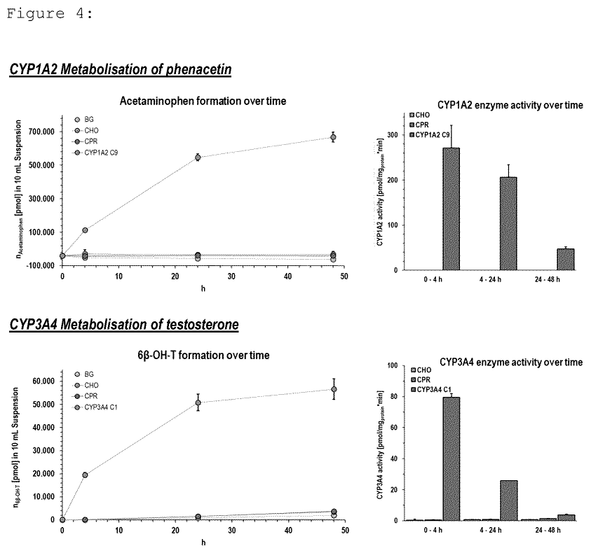

Metabolization studies to demonstrate CYP-specific conversion of prototype substances have been conducted to date with the mono-CYP CHO cell clones CHO-CPR/CYP1A2 K9 (phenacetin) and CHO-CPR/CYP3A4 K1 (testosterone) compared with the parental cell lines on a laboratory scale (suspension cultures up to 10 mL) over a maximum period of 48 hours. The cells were pretreated according to the current optimized culture model and metabolized in cell culture medium. The results of the corresponding supernatant analysis by HPLC are shown in FIG. 4. Based on normalization to measured standards of the respective main metabolite, yields achieved in the suspension cultures after 48 hours of reaction time could be extrapolated on a μg scale (CYP1A2: m_{(acetaminophen)}=101±4 μg; CYP3A4: m_{(6β-OH-T)}=17.3±1.4 μg). At the same time, initial detailed insights into enzyme kinetics were gained, which will be incorporated into further optimizations of the cell culture model and the conversion protocol.

### Example 4 a

### Specific Mono-CYP Enzyme Activity in Mono-CYP CHO Microsomes

### Commercial Glo Assays (Promega)

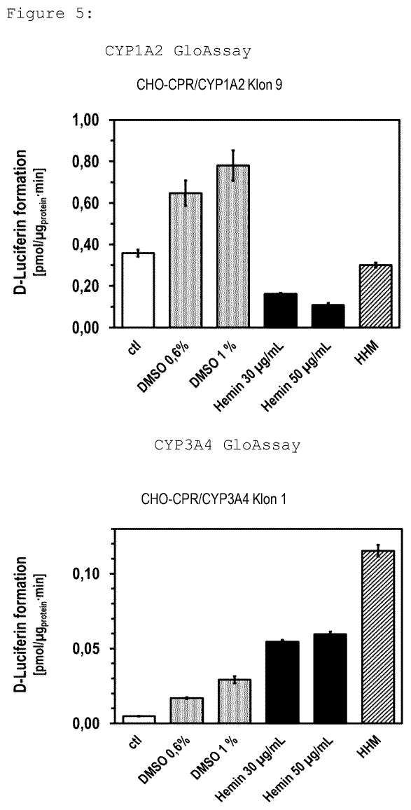

Enzyme activity studies were performed as part of the optimization of a cell pretreatment with cofactors and the microsome isolation protocol (FIG. 5).

### Example 4 b

Transformation Studies with Prototype Substances

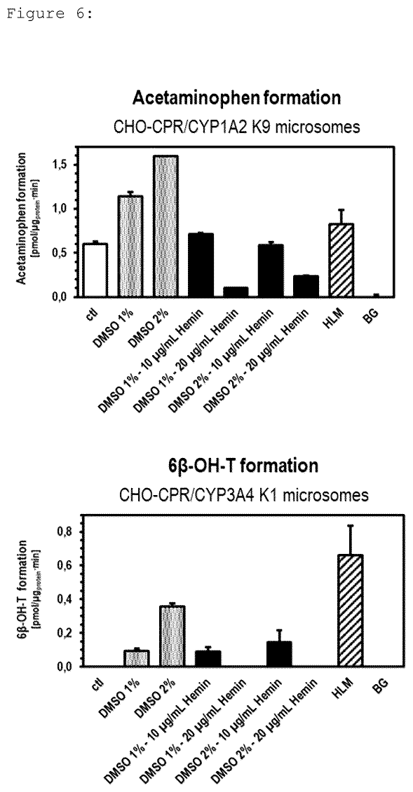

The microsomes of the mono-CYP CHO clones (CHO-CPR/CYP1A2 K9, CHO-CPR/CYP3A4 K1 and CHO-CPR/CYP2D6 C1, C9 &amp; C18) were also used for the “ ” metabolism of the prototype substances phenacetin (CYP1A2), testosterone (CYP3A4) and luciferin-ME EGE (CYP2D6) in order to gain insights into enzyme activity and yields of major metabolites formed (FIG. 6). The yields determined using standards of the main metabolites of phenacetin and testosterone in the 200 μL metabolism preparations were in the range of 100-200 ng and were partly comparable to HLM.

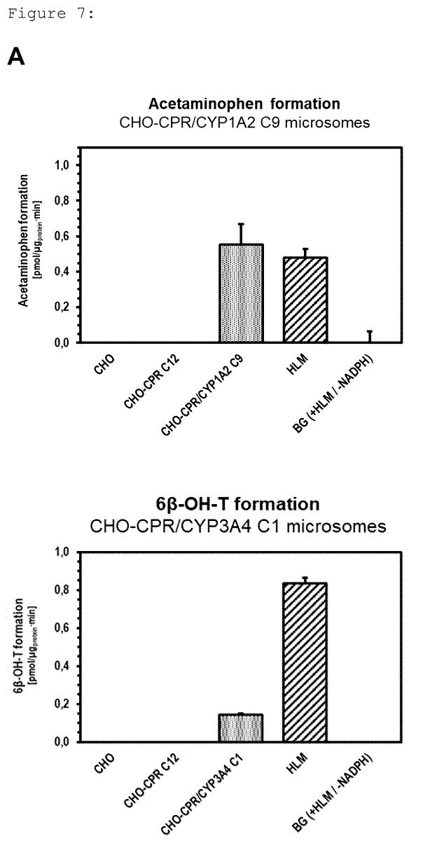

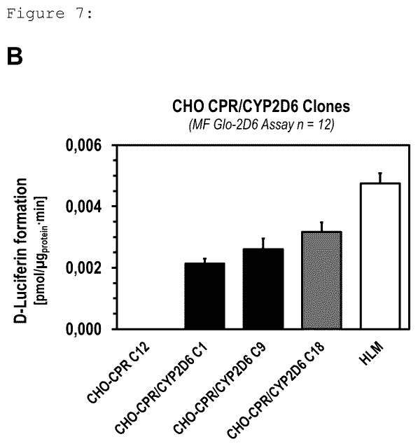

Direct comparison of mono-CYP CHO microsomes for CYP1A2, CYP3A4, and CYP2D6 with microsomes from the parental cell lines CHO-K1 and/or CHO-CPR K12 showed no detectable CYP-based background conversion in the parental microsomes based on conversion studies with prototypical substrates (FIG. 7). The yields of the main metabolites of phenacetin (acetaminophen) and testosterone (6β-hydroxytestosterone) in the mono-CYP microsomes in the 200 μL preparations were in the range of up to 100 ng, as previously reported.

### Example 5

### Example of the Detection of the Involvement of a Specific CYP450 Enzyme in the Metabolism of a Test Substance or Active Ingredient (CYP Screening Using the Example of Bupropion Metabolism)

To determine whether a test substance, e.g., bupropion (BUP), is metabolized by a specific CYP450 enzyme, conversion studies are conducted with isolated microsomes from CHO clones with recombinant CPR and different recombinant CYP activity (e.g., CYP3A4, CYP1A2, CYP2B6). Bupropion is a norepinephrine and dopamine reuptake inhibitor used to treat depression, seasonal affective disorder, and smoking cessation ( ). The use of microsomes in this test system has the advantage that primary and secondary metabolization outside the CYP-dependent phase 1 biotransformation is avoided, as the corresponding phase 2 biotransformation enzymes are absent in this predominantly ER-based cell fraction. After the conversion reaction, the aqueous supernatant of each CYP450 sample is examined using appropriate analytical methods (HPLC, LC-MS, etc.). This detects the reduction in the test substance caused by metabolism by a specific CYP enzyme and the formation of metabolites, and their identity is determined using mass spectrometry if necessary.

In accordance with the standardized protocol, 30 μg of microsomes with specific CYP450 activity (total protein amount) are mixed with a NADPH regeneration system (consisting of NADP+, glucose-6-phosphate, and the enzyme glucose-6-phosphate dehydrogenase) and the test substance (final concentration of 0.5 mM) in a 200 μL preparation for 60 minutes at 37° C. After the reaction, the samples are mixed 1:1 with ice-cold acetonitrile, shaken, and the proteins are precipitated from the solution by centrifugation (15 min, 16,000×g). This step stops further metabolism and removes proteinogenic components from the sample that would interfere with HPLC analysis.

The test substance remaining in a sample is quantified and metabolite profiles are created by subsequent HPLC analysis, initially with DAD detection. The HPLC setup consists of a modular (U) HPLC system from the Nexera series (LC-40, SHIMADZU, Kyoto, Japan) with a ZORBAX SB-C18 column (Agilent Technologies, Santa Clara, CA, US), which is equilibrated at 25° C. and. To detect the remaining test substance BUP and possible metabolites, 20 μL of sample in the mobile phase, consisting of 45% methanol and 55% KH2PO4 buffer (50 mM, pH 5.5), are separated isocratically at a flow rate of 0.8 mL/min for 25 minutes. Bupropion (BUP) and the most prominent CYP450-formed main metabolite hydroxybupropion (HBUP) are detected based on their elution time at 254 and 214 nm, respectively. In this example, the quantified substances are determined using standard solutions of commercially available references in the range of 0.1-1 mM for BUP and 1-75 μM for HBUP. BUP and HBUP are dissolved in methanol to prepare the stock solutions. If unknown signals (peaks) appear in the chromatogram of the respective CYP450 microsomes, which would indicate the formation of further metabolites, the samples are measured again using LC-MS and the chemical identity of unknown signals is determined based on their fragmentation pattern in correlation with theoretical predictions of the fragmentation of chemically possible metabolites.

In the case of the test substance bupropion, a reduction in the original amount of substance in the sample and the formation of HBUP are detectable exclusively in microsomes with specific CYP2B6 activity. Microsomes with specific CYP1A2 or CYP3A4 activity show neither a reduction in the original amount of bupropion nor the formation of metabolites of this test substance.

### Example 6

Example of the Detection of Drug Interactions with the CYP450 Enzyme System.

The example cited describes the detection of a drug interaction through inhibition of CYP3A4 enzyme activity by ketoconazole. Ketoconazole is a drug used, among other things, to treat or prevent fungal infections such as blastomycosis, candidiasis, coccidioidomycosis, histoplasmosis, chromomycosis, and paracoccidioidomycosis. One of the interactions of this drug is that it is a strong inhibitor of CYP3A4 enzyme activity. In the human body, this can inhibit the biotransformation of drugs that are primarily metabolized by CYP3A4, leading to an increased and/or prolonged effect of these drugs. Conversely, prodrugs that are activated by CYP3A4 (e.g., tamoxifen) may have a reduced and/or delayed effect when ketoconazole is used. The example of ketoconazole demonstrates the great benefit of in vitro test systems, such as the system according to the invention, for clarifying such drug interactions at an early stage.

To investigate the influence of ketoconazole on CYP3A4 activity, conversion studies are performed with isolated microsomes from CHO clones with recombinant CPR and specific recombinant CYP3A4 activity (e.g., CHO-CPR/CYP3A4 C1) in the presence and absence of ketoconazole. Differences in CYP3A4 activity are investigated either using commercially available enzyme activity assays (e.g., CYP3A4-Glo Assay from Promega) or analysis of the aqueous supernatants using appropriate analytical methods (HPLC, LC-MS, etc.). The latter is described in the following example.

Analogous to Example 5, 30 μg of microsomes (total protein amount) from the cell clone CHO-CPR/CYP3A4 C1 with specific CYP3A4 activity are incubated with a NADPH-e regeneration system (consisting of NADP+, glucose-6-phosphate, and the enzyme glucose-6-phosphate dehydrogenase) the prototypical CYP3A4 substrate testosterone (final concentration of 0.5 mM) and various concentrations of the test substance ketoconazole (range between 5 and 100 μM) in a 200 μL preparation for 60 min at 37° C. After the reaction, the samples are mixed 1:1 with ice-cold acetonitrile, shaken, and the proteins are precipitated from the solution by centrifugation (15 min, 16,000×g).

The quantification of the amount of testosterone remaining in a sample and the detection of the main metabolite 6β-hydroxytestosterone (6β-OH-T) formed by CYP3A4 is performed by subsequent HPLC analysis with DAD detection. The HPLC setup consists of a modular (U) HPLC system from the Nexera series (LC-40, SHIMADZU, Kyoto, Japan) with a ZORBAX SB-C18 column (Agilent Technologies, Santa Clara, CA, US), which is equilibrated at 25° C. For quantitative detection of residual substrate and formed 6β-OH-T, 20 μL of sample is separated in a gradient of KH2PO4 buffer (10 mM, pH 3, mobile phase A) and acetonitrile (mobile phase B) at a flow rate of 1 mL/min. The metabolites are detected based on the elution time with DAD (SPD-M10AvP) at 245 nm. The gradient is as follows: 0-3 min 30% B, 20.5 min 55% B, 22.5 min 55% B, 23 min 95% B, 26 min 95% B, 26.5 min 20% B, 29 min 20% B, 30 min 30% B. In this example, the quantification of analytes is performed using standard solutions of commercially available references in the range of 0.1-1 mM for testosterone and 1-75 UM for 6β-OH-T. From the chromatograms of the measured samples, the signals (peaks) for testosterone and 6β-OH-T are quantitatively evaluated using the standard curves, and the decreasing CYP3A4 activity with increasing concentration of ketoconazole ( ) is detected. It can be seen that the amount of 6β-OH-T formed via CYP3A4 decreases with increasing ketoconazole concentrations. This demonstrates that there is a drug interaction between ketoconazole and testosterone, in that the CYP3A4 metabolism of testosterone is inhibited by ketoconazole.

FIG. 1: Recombinant CPR expression and activity in selected CHO-CPR cell clones. CPR expression was analyzed at the mRNA level by RT-qPCR (A) and at the protein level by Western blotting (B). The increased CPR expression was also confirmed by indirect immunofluorescence (CPR in red, cell nuclei in blue) (D). CPR activity was determined from isolated microsomal fractions of recombinant CPR-expressing CHO clones compared to HepG2, HHM, and parental CHO (C) (HHM=human hepatocyte microsomes; mRNA expression with n=6 and CPR activity data with n=11 shown as mean±standard deviation; p<0.05 with * compared to CHO; * compared to HHM; * compared to HepG2; * compared between CPR clones).

FIG. 2: Recombinant CYP and CPR/CYP gene and protein expression in modified CHO clones. Selected CHO clones with CYP1A2, CYP2B6, CYP3A4, and CYP2D6 modification that express the respective CYP most strongly at the mRNA (A) and protein (B) levels are shown. Clear and specific overexpression of the respective transfected CYP enzyme was detected in all clones. Overexpression of CPR was also detectable in all CHO-CPR/CYP clones (mRNA expression data shown as mean±standard deviation; n=3).

FIG. 3: Mono-CYP activity in modified CHO clones with artificial phase 1 enzyme expression.
(A) Comparative analysis of the specific CYP activity of generated mono-CYP CHO clones for CYP1A2, CYP2B6, and CYP3A4 with and without overexpression of CPR, compared to the parental cell lines CHO-K1 and CHO-CPR clone 12 (data shown as mean±standard deviation; * p<0.05 compared to CHO; #compared between CYP clones; n=12 for CHO-based clones; n=6 for HepG2-CYP overexpressing positive controls).
(B) Comparative analysis of the specific CYP activity of generated mono-CYP CHO clones for CYP2D6 and CYP2C19 with overexpression of CPR, compared to the parental cell lines CHO-K1 and CHO-CPR clone 12 (Data presented as mean±standard deviation; n=3; for HepG2-CYP overexpressors as positive control).

Metabolization of prototypical substrates in mono-CYP CHO suspension cultures. Comparative analysis of the specific CYP activity of generated mono-CYP CHO clones with CPR overexpression compared to the parental cell lines CHO-K1 and CHO-CPR clone 12 (Data presented as mean±standard deviation; BG=conversion mixture without cells, CHO=parental cell line CHO-K1, CPR=parental cell line CHO-CPR clone 12; HPLC analysis with n=4 based on double quantification of two independent metabolism experiments).

FIG. 5: Mono-CYP activity in modified CHO clones after pretreatment of the cells with cofactors. Comparative analysis of the specific CYP activity of generated mono-CYP CHO clones with CPR overexpression after pretreatment of the cells with hemin (vehicle: DMSO). Untreated HHM served as a physiological positive control (reactions with 1 μg protein e for 10 min in 50 μL; data shown as mean±standard deviation; n=6 from two independent experiments).

FIG. 6: Mono-CYP activity in modified CHO clones after pretreatment of the cells with cofactors. Comparative analysis of the specific CYP activity of generated mono-CYP CHO microsomes with CPR overexpression after pretreatment of the cells with DMSO and combinations of DMSO with hemin. Untreated HLM served as physiological positive control (reactions with 30 μg protein for 30 min in 200 μL; data presented as mean±standard deviation; n=2).

FIG. 7: (A) Mono-CYP activity in CPR and CYP1A2 and CYP3A4-transfected CHO clones compared with parental cell lines. Comparative analysis of the specific CYP activity of mono-CYP CHO microsomes produced with parental cell lines (CHO and CHO-CPR C12) after pretreatment of the cells with DMSO. Untreated HLM served as physiological positive control (reactions with 30 μg protein for 30 min in 200 μL; data presented as mean±standard deviation; n=6). (B) Mono-CYP activity in CPR and CYP2D6-transfected CHO clones compared with the parental cell line CHO-CPR C12 for selection of suitable clones with CYP2D6 activity. Untreated HLM served as physiological positive control (reactions with 1 μg protein for 10 min in 50 μL in a Glo-CYP2D6 assay; data shown as mean±standard deviation; n=12).

FIG. 8 shows an example of an expression vector.

### REFERENCES

1. Elaut G, Henkens T, Papeleu P, Snykers S, Vinken M, Vanhaecke T, Rogiers V (2006): Molecular mechanisms underlying the dedifferentiation process of isolated hepatocytes and their cultures. Current drug metabolism 7 (6): 629-60;
2. Brandon E F, Raap C D, Meijerman I, Beijnen J H, Schellens J H. An update on in vitro test methods in human hepatic drug biotransformation research: pros and cons. Toxicology and applied pharmacology. 2003; 189 (3): 233-46.
3. Nauen R, Zimmer C T, Vontas J. Heterologous expression of insect P450 enzymes that metabolize xenobiotics. Current Opinion in Insect Science. 2021; 43:78-84.
4. Christensen H, Mathiesen L, Postvoll L W, Winther B, Molden E. Different enzyme kinetics of midazolam in recombinant CYP3A4 microsomes from human and insect sources. Drug metabolism and pharmacokinetics. 2009; 24 (3): 261-8.
5. Kumondai M, Hishinuma E, Rico EMG, Ito A, Nakanishi Y, Saigusa D, et al. Heterologous expression of high-activity cytochrome P450 in mammalian cells. Scientific reports. 2020; 10 (1): 1-13.
6. Jacob J, Raab G, Soballa V, Schmalix W A, Grimmer G, Greim H, et al. Cytochrome P450-mediated activation of phenanthrene in genetically engineered V79 Chinese hamster cells. Environmental toxicology and pharmacology. 1996; 1 (1): 1-11.
7. Krebsfaenger N, Mürdter T E, Zanger U M, Eichelbaum M F, Doehmer J. V79 Chinese hamster cells genetically engineered for polymorphic cytochrome P450 2D6 and their predictive value for humans. ALTEX-Alternatives to animal experimentation. 2003; 20 (3): 143-54.
8. Scheuenpflug J, Krebsfänger N, Doehmer J. Heterologous co-expression of human cytochrome P450 1A2 and polymorphic forms of N-acetyltransferase 2 for studies on aromatic amines in V79 Chinese hamster cells. Alternatives To Laboratory Animals. 2005; 33 (6): 561-77.
9. Ding S, Yao D, Burchell B, Wolf C R, Friedberg T. High levels of recombinant CYP3A4 expression in Chinese hamster ovary cells are modulated by coexpressed human P450 reductase and hemin supplementation. Archives of Biochemistry and Biophysics. 1997; 348 (2): 403-10.
10. Ding S, Yao D, Deeni Y Y, Burchell B, Wolf C R, Friedberg T. Human NADPH-P450 oxidoreductase modulates the level of cytochrome P450 CYP2D6 holoprotein via haem oxygenase-dependent and-independent pathways. Biochemical Journal. 2001; 356 (2): 613-9.
11. Deeni Y Y, Ibbotson S H, Woods J A, Wolf C R, Smith G. Cytochrome P450 CYP1B1 interacts with 8-methoxypsoralen (8-MOP) and influences psoralen-ultraviolet A (PUVA) sensitivity. Plos one. 2013; 8 (9): e75494.
12. Deeni Y Y, Ibbotson S H, Woods J A, Wolf C R, Smith G. Cytochrome P450 CYP1B1 interacts with 8-methoxypsoralen (8-MOP) and influences psoralen-ultraviolet A (PUVA) sensitivity. Plos one. 2013; 8 (9): e75494.

1. A mammalian cell comprising, after transfection or transduction, polynucleotides stably integrated into the genome encoding at least one cytochrome P450 and NADPH cytochrome P450 oxidoreductase.

2. The mammalian cell according to claim 1, wherein the mammalian cell exhibits no or low background activity of endogenous cytochrome P450 for the at least one cytochrome P450 and NADPH cytochrome P450 oxidoreductase.

3. The mammalian cell according to claim 2, wherein the low background activity of endogenous cytochrome P450 is either below a detection limit using a HPLC method for detected metabolites and/or is at most 5% of a specific activity in pmol/mg/min for the at least one cytochrome P450 and NADPH cytochrome P450 oxidoreductase.

4. A cell line comprising the mammalian cell according to claim 1.

5. A microsome obtained from the mammalian cell according to claim 1.

6. The microsome according to claim 5, wherein at least 100 μg of protein is contained in 10⁷ mammalian cells.

7. Suspension cells obtained from a mammalian cell according to claim 1.

8. A test system comprising cytochrome P450 (CYP) and NADPH cytochrome P450 oxidoreductase (CPR, POR) from one or more mammalian cells according to claim 1.

9. The test system according to claim 8, wherein the test system is configured to detect metabolites from substances.

10. The test system according to claim 8, wherein the test system is configured to detect a CYP450 enzyme or enzymes responsible for the biotransformation of at least one test substance, wherein an enzymatic conversion of at least one test substance with one or more CYP450 enzymes takes place.

11. The test system according to claim 8, wherein the test system is configured to detect an interaction of at least one test substance with at least one CYP450 enzyme.

12. The mammalian cell according to claim 1, wherein the mammalian cell is selected from the group consisting of non-hepatic cells, HEK293 cells, CHO cells, HeLa cells, BHK cells, and L929 cells.

13. The mammalian cell according to claim 1, wherein the cytochrome P450 is selected from the group consisting of CYP1A1, CYP1A2, CYP1B1, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3A4, CYP3A5, CYP3A7, CYP3A43, CYP4A11, CYP4A22, CYP4B1, CYP4F2, CYP4F3, CYP4F8, CYP4F11, CYP4F12, CYP4F22, CYP4V2, CYP4X1, CYP4Z2, CYP5A1, CYP7A1, CYP7B1, CYP8A1, CYP8B1, CYP11A1, CYP11B1, CYP11B2, CYP17A1, CYP19A1, CYP20A1, CYP21A1, CYP21A2, CYP24A1, CYP26A1, CYP26B1, CYP26C1, CYP27A1, CYP27B1, CYP27C1, CYP39A1, CYP46A1, and CYP51A1.

14. The mammalian cell according to claim 1, wherein cytochrome P450 and NADPH cytochrome P450 oxidoreductase are overexpressed.

15. The mammalian cell according to claim 1, wherein a polynucleotide integrated into the mammalian cell genome is contained in an expression vector encoding at least one cytochrome P450 and NADPH cytochrome P450 oxidoreductase and is controlled by at least one promoter.

16. The test system according to claim 9, wherein the substances comprise xenobiotics, active substances, and drugs.


SEQUENCE LISTING (8 sequences)

SEQ ID NO: 1 - DNA, 20 bp, synthetic construct
  source 1..20: mol_type = other DNA
aaggcggtgcccacatctac

SEQ ID NO: 2 - DNA, 20 bp, synthetic construct
  source 1..20: mol_type = other DNA
tagcggcccttggtcatcag

SEQ ID NO: 3 - DNA, 20 bp, synthetic construct
  source 1..20: mol_type = other DNA
ctggagaccttccgacactc

SEQ ID NO: 4 - DNA, 20 bp, synthetic construct
  source 1..20: mol_type = other DNA
agggcttgttaatggcagtg

SEQ ID NO: 5 - DNA, 20 bp, synthetic construct
  source 1..20: mol_type = other DNA
ctctccatgacccacactac

SEQ ID NO: 6 - DNA, 20 bp, synthetic construct
  source 1..20: mol_type = other DNA
tgttgggggtattttgccca

SEQ ID NO: 7 - DNA, 21 bp, synthetic construct
  source 1..21: mol_type = other DNA
gtggggcttttatgatggtca

SEQ ID NO: 8 - DNA, 23 bp, synthetic construct
  source 1..23: mol_type = other DNA
gcctcagatttctcaccaacaca